(19) United States
(12) Patent Application Publication
(10) Pub. No.: US 2015/0133796 A1
(43) Pub. Date: May 14, 2015

(54) SYSTEMS AND METHODS FOR USING PHYSIOLOGICAL INFORMATION

(71) Applicant: CardioMEMS, Inc., Atlanta, GA (US)

(72) Inventor: Jay Yadav, Atlanta, GA (US)

(21) Appl. No.: 14/534,834

(22) Filed: Nov. 6, 2014

Related U.S. Application Data
(63) Continuation of application No. 13/205,459, filed on Aug. 8, 2011, now abandoned.
(60) Provisional application No. 61/371,391, filed on Aug. 6, 2010.

(51) Int. Cl. A61B 5/00; A61B 5/0215; A61B 5/029; A61B 5/0205; G06F 17/30; A61B 5/02

(52) U.S. Cl. CPC A61B 5/4848; G06F 17/30424; A61B 5/02007; A61B 5/029; A61B 5/0205; A61B 5/0215; A61B 5/7275; A61B 5/02438
USPC 600/484; 707/769; 600/481; 600/526; 600/508; 600/529; 600/485; 600/486

(57) ABSTRACT

Systems and methods using a database of physiological information for the design, development, testing and use of therapeutics. In one aspect, the physiological information can include at least one of: hemodynamic monitoring information, pulmonary arterial pressure, cardiac output, heart rate, respiratory rate, peripheral vascular resistance, total peripheral resistance or dicrotic notch information. Optionally, the cardiovascular physiology information can include ambulatory physiological information.

Pursuant to 35 U.S.C. §119(e), this application claims priority to the filing date of U.S. Provisional Patent Application Ser. No. 61/371,391, filed Aug. 6, 2010; the disclosure of which application is herein incorporated by reference.

### FIELD OF THE INVENTION

The present invention relates generally to health care and particularly to therapeutic regimens. More specifically, to methods and systems for using physiological information for the design, development, testing and use of therapeutics in treating patients.

### DESCRIPTION OF THE RELATED ART

Biology has undergone a change so fundamental that it has been compared to the industrial revolution of the 19th century and the advances in quantum physics in the 20th century. For example, the complete sequencing of the human genome and of the genomes of many microbes and plants has given rise to genomics, the discipline defined as the study of the structure and function of large number of genes undertaken in a simultaneous fashion. While the value of genomics as a basic tool for biological research has been clearly demonstrated, the impact on drug discovery remains unrealized.

Drug development is rife with failures, many of them expensive and deep into regulatory approval pipelines. In spite of the availability of numerous targets for drug discovery, the overall success rate of the process remains abysmally low. There are three main reasons for low success rates in the conversion of vast amounts of genomics information to viable products: lack of clear criteria for target validation; hits to leads decisions based on potency and selectivity against molecular targets, with limited physiological information; and nonviable leads due to poor adsorption, undesirable metabolism, toxicity, or unacceptable side effects.

Drug development programs typically rely on in vitro screening assays and subsequent testing in appropriate animal models to evaluate drug candidates prior to conducting clinical trials using human subjects. Screening methods currently used are generally difficult to scale up to provide the high throughput screening necessary to test the numerous candidate compounds generated by traditional and computational means. Moreover, current studies involving cell culture systems and animal model responses frequently don't accurately predict the responses and side effects observed during human clinical trials. Further, conventional methods for assessing the effects of various agents or physiological activities on biological materials, in both in vitro and in vivo systems, generally are not highly sensitive or informative.

With each drug costing an estimated $1 billion to develop, earlier detection of improved candidates and better designed studies and more carefully chosen patient populations can result in enormous savings by avoidance of failure. Better and earlier information can improve the development outcomes of therapeutics for a host of diseases such as cardiovascular diseases, which is still the number one cause of death in the United States, ocular, urological, neurological and gastroenterological diseases. Improvements in therapeutic applications for such diseases can have a large positive impact and are highly desirable.

### SUMMARY

The systems and methods described herein use physiological information for the design, development, testing and use of therapeutics. In one aspect, exemplary systems and methods are described that are suitable for developing a therapeutic using a database of physiological information. In one aspect, it is contemplated that hemodynamic information from one or more subjects can be used for therapeutic development. In another aspect, the database of physiological information can comprise cardiovascular physiology information from one or more subjects. In various aspects, the cardiovascular physiology information can comprise at least one of: hemodynamic monitoring information, pulmonary arterial pressure, cardiac output, heart rate, respiratory rate, peripheral vascular resistance, total peripheral resistance or dicrotic notch information. Optionally, the cardiovascular physiology information can comprise ambulatory cardiovascular information.

In one aspect, one or more cardiovascular physiology information inputs can be remotely obtained by use of wireless technologies, for example. In another aspect, it is contemplated that one or more cardiovascular physiology information inputs can be obtained from an implanted sensor, such as, for example and without limitation, a pressure sensor that is implanted in a desired location within the patient. In one example and without limitation, the desired location can be a selected portion of the subject's pulmonary artery. Of course, it is contemplated that physiology information suitable for use in the system and method described herein can be supplied or otherwise employed from conventional ocular, neurological, urological and gastroenterological systems.

In one aspect, the development of a therapeutic can comprise prospectively guiding development of the therapeutic using a database of physiological information. As used throughout, the term “therapeutic” is used interchangeably with the term “therapeutic agent.” In one aspect, the prospective guidance of development of the therapeutic can comprise designing the therapeutic and, optionally, can further comprise designing a testing protocol for the therapeutic. In some aspects, patients can be chosen for a clinical trial based on the physiological data. Furthermore, the prospective guiding development of the therapeutic can comprise modeling predicted characteristics of a therapeutic.

In one aspect, the systems and methods described herein can optionally be used to predict characteristics of a therapeutic comprising at least one of efficacy, drug-drug interaction, safety, adverse events or dosing. In another aspect, the development of a therapeutic can comprise prospectively guiding development of the therapeutic using the database of physiological information can comprise using the database to meet regulatory requirements.

In other aspects, also provided are systems and methods for predicting an effect of a candidate therapeutic agent on a hemodynamic parameter of a patient. In one aspect, the systems and methods described herein can comprise providing at least one database including hemodynamic data, which can comprise a plurality of hemodynamic values that can be measured in one or more subjects.

In one aspect, a candidate therapeutic agent for administration to a patient can be identified and all or a selected subset of the hemodynamic data can be correlated with the candidate therapeutic agent to indicate a predicted change in one or more hemodynamic values in the patient that would result from administration of the candidate agent. In this aspect, the predicted change can be used to indicate the predicted effect of the candidate agent on the hemodynamic parameter of the subject.

In a further aspect, therapeutic agents can be designed by determining a change to a hemodynamic parameter of a subject or an expected change resulting from administration of the therapeutic agent. In this aspect, the change or expected change in the hemodynamic parameter can be used to design a therapeutic agent. For example, if the change or expected change is desirable, the therapeutic agent can be optionally modified to increase the magnitude, onset or duration of the change. In another example, if the change or expected change is undesirable, the therapeutic agent can be optionally modified to decrease the magnitude, onset or duration of the change.

In another aspect, the systems and methods can also comprise identifying a subject based on a specified hemodynamic response to a therapeutic agent. In one aspect, characteristics of the subject that indicate an increased likelihood that the subject will have the specified hemodynamic responses can be determined and, optionally, the identified subject or a plurality of subjects having the same or similar determining characteristics can be selected to participate in a clinical trial or study for the therapeutic agent or, alternatively, the identified subject or subjects can be selectively excluded from the clinical trial or study. In one aspect, the systems and methods can screen populations to identify subpopulations for study that have a common physiological profile characteristic such as the responsiveness of various physiological parameters, including but not limited to, hemodynamic parameters.

In one aspect, the systems and methods can comprise developing a therapeutic agent or regimen for administering the therapeutic agent. For example, a change to a hemodynamic parameter of a subject or an expected change resulting from administration of the therapeutic agent can be determined and the determined change or expected change in the hemodynamic parameter can be used to develop the therapeutic agent or regimen. In is also contemplated that the systems and methods can comprise assessing the safety or efficacy of a therapeutic agent. In this example, a change to a hemodynamic parameter of a subject or an expected change resulting from administration of the therapeutic agent can be determined and the determined change or expected change in the hemodynamic parameter can be used to assess the efficacy of the therapeutic agent.

In one aspect, the systems and methods can comprise assessing an effect of a therapeutic agent on a hemodynamic parameter of a subject and can comprise providing at least one database that comprises hemodynamic data comprising a plurality of hemodynamic values measured in one or more subjects that had each been administered a therapeutic agent.

In one aspect, a change in one or more of the measured hemodynamic values resulting from the administration of the therapeutic agent can be identified, the change indicating an effect of the therapeutic agent on the hemodynamic parameter of the subject. In one aspect, the hemodynamic data can comprise at least one hemodynamic value measured in a subject prior to administration of the therapeutic agent, at least one hemodynamic value measured in a subject concurrent with administration of the therapeutic agent, and/or at least one hemodynamic value measured in a subject subsequent to administration of the therapeutic agent. Optionally, the hemodynamic data comprises at least one hemodynamic value measured in a subject prior to administration of the therapeutic agent and at least one hemodynamic value measured in a subject subsequent to administration of the therapeutic agent. In some aspects, one or more additional therapeutic agents are administered to the subject prior to, concurrently with, or subsequent to the therapeutic agent.

In one aspect, the therapeutic agent can be modified to increase the indicated effect. For example, if the indicated effect is desired, the structure of the therapeutic agent can be modified to increase or decrease the desired degree of the indicated effect. In one aspect, if the indicated effect is not desirable, then the structure of the therapeutic agent can be modified to decrease the indicated effect.

In another aspect, an administration characteristic of the therapeutic agent can be modified to increase or decrease the desired degree of the indicated effect. In one aspect, the administration characteristic can be selected from the group comprising at least one of: dosage amount, number of doses, timing of doses, route of administration, and/or total dosage. In one aspect, when the indicated effect is to be increased or decreased, one or more portions of the therapeutic agent responsible for the indicated effect can be determined. In a further aspect, a second therapeutic agent including the one or more portions of the therapeutic agent responsible for the indicated effect can be designed.

In another aspect, the indicated effect can used to assess safety of the therapeutic agent for administration to a mammal or population thereof. In various aspects, the indicated effect can be used to assess at least one of the toxicity and efficacy of the therapeutic agent for administration to a mammal or population thereof. In one aspect, the toxicity can be, without limitation, cardiac toxicity. In another aspect, the indicated effect can also be used to predict the effect or effects of the therapeutic agent or agents having the same or similar pharmacological characteristics on the hemodynamic parameter or on a hemodynamic parameter of a mammal. In some aspects, the indicated effect is used to determine an end point for a clinical trial.

In one aspect, the method and system can further comprise determining one or more characteristic of the subject, such as, for example and without limitation, a physical, physiologic, metabolic, chronological, disease state, drug administration history, medical history, or genetic characteristic. In one aspect, the characteristic can be correlated with the indicated effect in the subject and the correlation of the characteristic and the indicated effect in the subject can be used to select one or more additional subjects for administration of the therapeutic agent or for a therapeutic agent having the same or similar indicated effect. In another aspect, the correlation of the characteristic and the indicated effect can also be used to select one or more additional subjects to participate in a clinical trial for the therapeutic agent or for a therapeutic agent having the same or similar indicated effect.

In one aspect, the correlation of the characteristic and the indicated effect in the subject can be used to select or modify a therapeutic regimen in the subject or in another subject having the same or similar characteristics. Such selection or modification can comprise selecting or modifying drug administration protocol, which can comprise, for example and without limitation, dosage of one or more therapeutic agent, selection of one or more therapeutic agent, combination of therapeutic agents, or timing of administration of one or more therapeutic agent. In one aspect, the indicated effect can also be used to alter a treatment protocol of a subject. For example and without limitation, the indicated effect can be used for determining whether to administer less of the therapeutic agent, administering more of the therapeutic agent, discontinuing use of the therapeutic agent, administering one or more additional agents, and the timing of administration of the agent.

In various aspects, the hemodynamic parameters can optionally be selected from the group comprising, for example and without limitation, heart rate, systolic blood pressure, diastolic blood pressure, mean blood pressure, stroke volume, cardiac output, peripheral vascular resistance, total peripheral resistance, and pulmonary arterial pressure. It is contemplated that the hemodynamic values measured in the subject can optionally be measured using an implantable sensor device, which can optionally measure hemodynamic parameters selected from the group comprising heart rate, systolic blood pressure, diastolic blood pressure, mean blood pressure, stroke volume, cardiac output, peripheral vascular resistance, total peripheral resistance and pulmonary arterial pressure.

In one aspect, the method or system can comprise a computer system comprising a memory on which is stored a database that contains high-fidelity physiological information obtained from a plurality of patients and that is correlated with a plurality of associated conditions; instructions for receiving from a user an inquiry about a therapeutic; instructions for determining a relationship between the therapeutic and one of the associated conditions, or the high-fidelity physiological information. In one aspect, the computer system can further comprise instructions for receiving a date stamp associated with the high-fidelity physiological information and with the ambulatory conditions and instructions for correlating the date stamps to develop associative information characterizing temporal relationships between the high-fidelity physiological information and the ambulatory conditions and store the associative information on the database. In one aspect, the inquiry about the therapeutic can be a design inquiry configured to prospectively predict success of the therapeutic based on a predicted physiological impact of the therapeutic and the high-fidelity physiological information. In one aspect, the high-fidelity physiological information is optionally obtained from an implanted sensor. In another aspect, the associated conditions can comprise ambulatory conditions.

Additional embodiments of the invention will be set forth, in part, in the detailed description, figures, and claims which follow, and in part will be derived from the detailed description, or can be learned by practice of the invention. It is to be understood that both the foregoing general description and the following detailed description are exemplary and explanatory only and are not restrictive of the invention as disclosed.

### BRIEF DESCRIPTION OF THE DRAWINGS

These and other features of the preferred embodiments of the invention will become more apparent in the detailed description in which reference is made to the appended drawings wherein:

FIG. 1 is a schematic of one embodiment of a therapeutic development system.

FIG. 2 .shows comparison between data received between a RHC “gold standard” and the measurements of a CARDIOMEMS pressure sensor. The top waveform shows some undesired whip or overshoot that is believed to exceed systolic and diastolic pressures and which can lead to error and uncertainty. The CARDIOMEMS sensor waveform on the bottom, in contrast, exhibits high-fidelity through its smooth and undistorted waveform.

FIG. 3 is a schematic of a front end computer system of the therapeutic development system of FIG. 1.

FIG. 4 is a flow chart of operation of the front end computer system of FIG. 3.

FIG. 5 is a schematic of a back end computer system of the therapeutic development system of FIG. 1.

FIG. 6 is a flow chart of operation of the back end computer system of FIG. 5.

FIG. 7 is an exemplary table of data entered into the front end computer system of FIG. 3.

FIGS. 8-10 are displays of selective data mined from a database of physiological information using the back end computer system of FIG. 5.

FIG. 11 is a schematic of another embodiment of a therapeutic development system.

FIG. 12 is a flow chart illustrating an exemplary method for developing a drug.

FIG. 13 is a flow chart illustrating an exemplary method for selecting and individual subject or a group of subjects for inclusion in or exclusion from a pharmaceutical trial.

FIG. 14 is a flow chart illustrating an exemplary method for guiding or facilitating the use of a therapeutic in a subject or population of subjects.

FIG. 15 is a flow chart illustrating exemplary use of physiological data design, development, testing and use of therapeutics.

FIGS. 16-20 are exemplary patient data charts.

### DETAILED DESCRIPTION

The present invention may be understood more readily by reference to the following detailed description, examples, drawings, and claims, and their previous and following description. However, before the present systems, and/or methods are disclosed and described, it is to be understood that this invention is not limited to the specific systems, and/or methods disclosed unless otherwise specified, as such can, of course, vary. It is also to be understood that the terminology used herein is for the purpose of describing particular aspects only and is not intended to be limiting.

As used in the specification and the appended claims, the singular forms “a,” “an” and “the” comprise plural referents unless the context clearly dictates otherwise. Thus, for example, reference to a “of pharmacological agent” can comprise two or more such of pharmacological agents unless the context indicates otherwise.

Ranges may be expressed herein as from “about” one particular value, and/or to “about” another particular value. When such a range is expressed, another aspect comprises from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description comprises instances where said event or circumstance occurs and instances where it does not.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The present invention may be understood more readily by reference to the following detailed description of preferred embodiments of the invention and the examples comprised therein and to the Figures and their previous and following description.

As used throughout, by a “subject” is meant an individual. The term “patient” comprises human and veterinary subjects.

The term “therapeutic” or “therapeutic agent” is used generally herein to refer to any compound, substance, process, method, device or other treatment, or combination thereof, that is ameliorative of or affected by or associated with the physiological information. A therapeutic can comprise a combination of pharmacological agents or substances, timing and amount of administration of the same or combination of those with various programs or routines, such as exercise or rehabilitation programs or routines. All therapeutic compounds disclosed herein with, and without, trade names can also comprise their respective active ingredients in other therapeutics, such as generic versions of the therapeutic, and combinations therapies containing such compounds. Example therapeutics can comprise, but are not limited to, cardiovascular, diabetes and non-steroidal anti-inflammatory (NSAID) agents. “It is contemplated that exemplary cardiovascular agents can comprise any therapeutically prescribed cardiovascular agent.”

Example diabetes agents can comprise, but are not limited to: Actos Oral, Amaryl Oral, ApidraSoloStarSubQ, AVANDAMET Oral, Avandaryl Oral, Avandia Oral, ByettaSubQ, Cozaar Oral, Diabeta Oral, Glucophage Oral, Glucotrol Oral, Glucovance Oral, Glynase Oral, Humulin R U-500 “Concentrated” Inj, Insulin Regular Human Inj, Insulin Regular Hum U-500 ConcInj, Lantus SubQMetaglip Oral, Micronase Oral, NPH Insulin Human RecombSubQ, Onglyza Oral, PramlintideSubQ, Prandimet Oral, Prandin Oral, Prccosc Oral, RIOMET Oral, Starlix Oral, SymlinPcn 120 SubQ, SymlinPcn 60 SubQ, Xenical Oral.

Example NSAIDs can comprise, but are not limited to: Aspirin (Anacin, Ascriptin, Bayer, Bufferin, Ecotrin, Excedrin), Choline and magnesium salicylates (CMT, Tricosal, Trilisate) Choline salicylate (Arthropan), Celecoxib (Celebrex), Diclofenac potassium (Cataflam), Diclofenac sodium (Voltaren, Voltaren XR), Diclofenac sodium with misoprostol (Arthrotec), Diflunisal (Dolobid), Etodolac (Lodine, Lodine XL), Fenoprofen calcium (Nalfon), Flurbiprofen (Ansaid), Ibuprofen (Advil, Motrin, Motrin IB, Nuprin), Indomethacin (Indocin, Indocin SR), Ketoprofen (Actron, Orudis, Orudis KT, Oruvail), Magnesium salicylate (Arthritab, Bayer Select, Doan's Pills, Magan, Mobidin, Mobogesic), Meclofenamate sodium (Meclomen), Mefenamic acid (Ponstel), Meloxicam (Mobic), Nabumetone (Relafen), Naproxen (Naprosyn, Naprelan), Naproxen sodium (Aleve, Anaprox), Oxaprozin (Daypro), Piroxicam (Feldene), Rofecoxib (Vioxx), Salsalate (Amigesic, Anaflex 750, Disalcid, Marthritic, Mono-Gesic, Salflex, Salsitab), Sodium salicylate (various generics), Sulindac (Clinoril), Tolmetin sodium (Tolectin), and Valdecoxib (Bextra).

The term “developing” or “development” as used herein in reference to therapeutics are broad terms that comprise, by way of example and not limitation, prospective design, or selection, of one or more potential therapeutic methods or compounds or retrospective study of one or more therapeutic methods or compounds or design of studies of such therapeutic methods or compounds. In this regard to develop or development of a therapeutic can comprise, for example, changes to an active ingredient or formulation and can also comprise, for example, study design for a therapeutic. Optionally, study design can be for a clinical trial and development of a study design that can comprise establishing trial metrics or trial durations based on physiological information.

The term “physiological information” comprises data or other information on the functional processes of living things, such as human bodies. Examples of physiological information comprise cardiovascular information such as hemodynamic parameters (e.g., cardiac output, peripheral vascular resistance, total peripheral resistance) or respiratory information, such as respiration rate and associated respiration volumes. In addition, physiology information can be employed from ocular, neurological, urological and gastroenterological systems.

Physiological information can also comprise combinations of mechanical and chemical parameters, such as pulse oximetry or blood oxygenation. Pulmonary artery pressure (PAP) is a particularly desired information set for clinician, scientists and other therapeutic developers. Right heart catheterization (RHC) to measure pulmonary artery pressure is the “gold standard” for determining cardiac hemodynamics. RHC, although yielding highly-desired PAP information, has drawbacks including invasiveness, infrequency of measurements, risk of infection and cost. As one skilled in the art will appreciate, RHC is particularly ill-suited for ambulatory measurements.

As defined herein, “ambulatory measurements” refers to measurements that are made in normal daily-living situations where the patient is not bedridden in a clinical setting. For example, sleeping (e.g., for studies and therapies of sleep apnea), eating and exercise activities at the home or work environments where RHC and other more invasive procedures are largely impractical and/or risky.

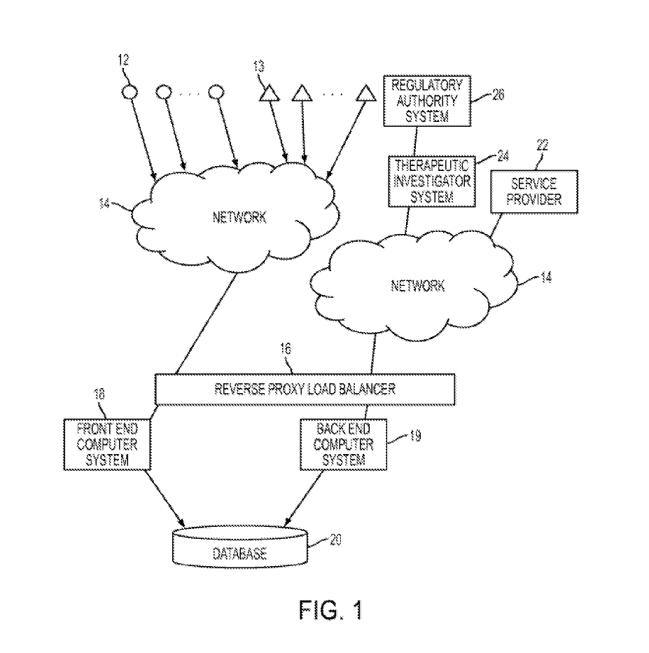

In one aspect, provided herein are systems and methods for using physiological information for therapeutic development. For example, hemodynamic information from one or more subject can be used for therapeutic development. Generally, embodiments comprise systems, processes and computer programs configured for developing a therapeutic using a database of physiological information. In one aspect, a therapeutic development system 10 of one embodiment of the present invention is shown in FIG. 1 and comprises a plurality of patient monitors 12, a plurality of healthcare personnel 13, one or more networks 14, a security system 16, a front end computer system 18, a back end computer system 19, a database 20, a service provider 22, a therapeutic investigator system 24 and a regulatory authority system 26.

In one aspect, the patient monitors 12 are preferably systems configured to sense physiological information in ways that enable effective use of the database 20 in therapeutic development. It is contemplated that characteristics of the respective physiological information can comprise high fidelity, long-duration, and/or remote sensing of patients in ambulatory environments. It is contemplated data of sufficiently high volumes can be sufficient to yield statistical differences needed to prospectively improve target identification, clinical trials, patient selection, regulatory protocol design and statistical differentiation of desired end points.

In one aspect, one exemplary effective system and sensor suitable for measurement of hemodynamic parameters is the CARDIOMEMS pressure sensor. As described by U.S. Pat. No. 7,699,059 entitled “Implantable Wireless Sensor” and U.S. Pat. No. 7,679,355 entitled “Communicating with an Implanted Wireless Sensor,” which patent publications, in their entireties, are hereby incorporated by reference into this application, these pressure sensors are MEMS-based pressure sensors that are configured to be implanted: in the pulmonary artery, more particularly in the distal pulmonary artery branch, with a RHC or as part of a graft, such as a AAA stent-graft, and the like. The CARDIOMEMS pressure sensor are further configured to be selectively energized with RF energy to return high-frequency, high-fidelity dynamic pressure information from a precisely-selected location within a patient's body. In one aspect, advantages of the CARDIOMEMS pressure sensor when used in therapeutic development are that: the system is wireless, the pressure sensor is non-invasive after initial implantation, the pressure sensor is small enough to be implanted in a desired range of lumens and locations within a patient, and the pressure sensor is permanent or can be implanted for prolonged durations.

Another advantage of the CARDIOMEMS pressure sensor is that it can make measurements during ambulatory activities away from the hospital that are more representative of living conditions of a patient who is going to use a therapeutic. Because the CARDIOMEMS pressure sensor is non-invasive after implantation, ambulatory use is provided and the CARDIOMEMS sensor can be selectively energized via an easy-to-use RF transmitter within an external, non-invasive device that energizes the sensor. In another aspect, the CARDIOMEMS pressure sensor is configured to communicate pressure data wirelessly to a node local to the patient that is configured to transmit the information over the network 14 to the front end computer system 18 with little or no involvement of the patient.

In another aspect, the patient group and data set yielded by the CARDIOMEMS pressure sensor is particularly large and dense. For example, in trials have been run for heart failure management with monitoring of cardiac hemodynamics for the treatment of heart failure in over 600 patients for more than 4 years (the HF study). In addition, the accumulated data is collected in “real time” at “remote” locations, which “real time” collection of data comprises physicians having almost instantaneous access to monitored data via transmission to a range of devices. These devices comprise, for example, a physician's PDA (e.g., BLACKBERRY®, RIM, Waterloo, ON) or access through a secure website. For “remote” collection of data, the monitoring occurs at the patient's home (or elsewhere) without geographic limitation with respect to the physician's location. In all, 244,835 patient days, with a mean of 445 days per patient and a maximum of 916 days, were recorded in the HF study. Total number of readings in the HF study exceeds 200,000. Worldwide, the numbers are even higher with 290,799 days total, a mean of 483 days per patient and a maximum of 1496 days. Treatment regimens were associated with statistically significant (p<0.05) drops in HF events (−21%; p=0.33), reduction of worsening HF (−36%, p=0.035) and reduction in HF events for class 3 patients (−41%; p=0.03).

In a different application, over 7,000 implants of the CARDIOMEMS pressure sensor have been performed with abdominal aortic aneurysm (AAA) stent-grafts and data has been accumulated over the course of the past years. Other applications of the implantable CARDIOMEMS pressure sensor comprise evaluation of portal hypertension, yielding information for drug therapies that couldn't previously be readily assessed. Measurements of portal hypertension can assess treatments for hepatitis, alcoholism and fatty liver disease. Systems and methods of embodiments of the system and methods described herein enable accumulation and use of such large volumes of data.

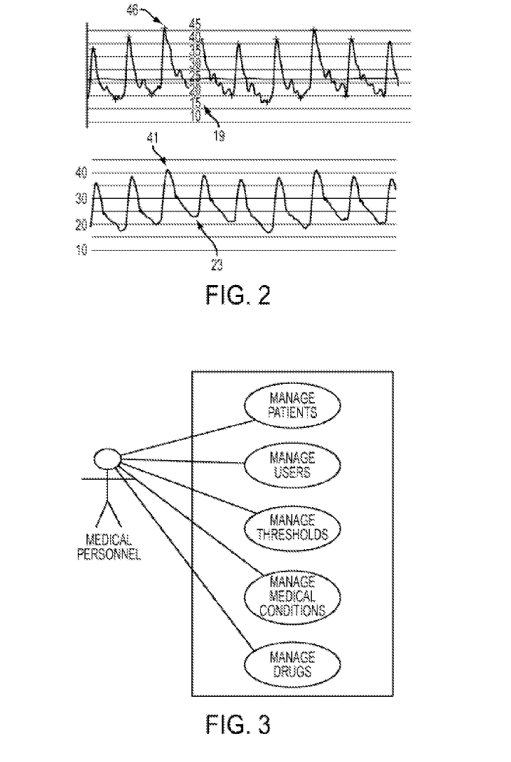

In one aspect, the data obtained with the CARDIOMEMS sensor are examples of physiological data in that it is “high fidelity.” CARDIOMEMS pressure sensor allows for sampling rates are at 2,000 samples per second without fluidic artifacts and can be collected without line occlusion, which is the tendency of the line of a RHC to affect the hemodynamic measurements, and a lack of distortion due to movement that occurs in invasive procedures with long leads or wires extending from the patient. The accuracy of the CARDIOMEMS sensor data are also aided by the addition of resistance effects to the basic Bernoulli model, using Windkessel principles. In another aspect, the CARDIOMEMS sensor data has been validated. In the aforementioned HF study, results were robust when compared to RHC data, within +/−10 mmHg at 95% limits over long (several hundred days) periods of time without any deterioration. A comparison between the RHC “gold standard” and the measurements of the CARDIOMEMS pressure sensor is shown in FIG. 2 wherein the waveform shows some undesired whip or overshoot that is believed to exceed systolic and diastolic pressures and which can lead to error and uncertainty. The CARDIOMEMS sensor waveform on the bottom, in contrast, exhibits high-fidelity through its smooth and undistorted waveform. For example, the dicrotic notch is clear and pronounced compared to the RHC gold standard.

In another aspect, the networks 14 shown on FIG. 1 could any type of network, including a local area network (LAN) or a wide area network (WAN), or the connection can be made to an external computer (for example, through the Internet using an Internet Service Provider). The advantage of the use of networks 14 in the present invention are that they enable the remote high-volume collection of ambulatory information from the monitoring systems 12 described above. The term “remote” as used herein comprises collection at locations other than hospital locations and in a manner that insubstantially interrupts the daily life activities of the patients. As a result, use of one or more networks 14 enables easy recruitment of larger, more diverse patient populations to be used in development of therapeutics in embodiments of the present invention. And, the data collected from those remote locations are more representative of the conditions in which the therapeutics must be safe and effective.

In one aspect, if used, the security system 16 of the present invention can comprises a reverse proxy load balancer with a 128 bit SSL encrypted with purposely limited functionality to protect confidential patient data. With this embodiment, specific ports can be opened to specific machines behind the security system 16, thereby minimizing access to the internal environment. Notably, in the illustrated embodiment all access to the front end computer system 18, back end computer system 19 and the physiological information database 20 is through this security system 16. Optionally, other safeguards comprise website timeout after predetermined period of time to prevent unauthorized intrusions from unmonitored workstations. Also, sensitive patient information can be encoded while at-rest within the physiological database 20. Therefore, even unauthorized access to the database 20 will not provide access to sensitive patient information.

The front end computer system 18 is configured, in the embodiment of FIG. 1, to act as a depot and gatekeeper to physiological information being communicated from the patient monitors 12 through the network 14 before it gets to the database 20. In particular, the front end computer system 18 is configured to host applications for the consultative addition of correlative information by the healthcare personnel 13. And, primarily, the front end computer system 18 is configured for the accessing and monitoring of data on the individual patient level to enable treatment. In another embodiment of the present invention, the front end computer system 18 has functions grouped as shown in FIG. 3 and including managing patients, users, thresholds, medical conditions and drugs. In one aspect, for example and without limitation, various embodiments of the front end computer system 18 can be configured to have one or more of the following functions: accept pressure and other physiological data from the patient monitoring systems 12; process reading data and determine a score based on an automated scoring algorithm; process readings with a passing score further and made available such reading to the appropriate medical personnel; que readings that do not pass the automated scoring for manual inspection by service providers 22; use a job queue sub-system to manage the processing of readings and other tasks within the system; provide an interface for medical personnel with appropriate permissions to manage users and site level preferences; provide an interface for medical personnel to import patient data from a thumb drive that was be created as part of the sensor implant procedure; provide an interface for medical personnel to create a patient record substituting for lost patient data: provide an interface for medical personnel to enter and modify patient information; provide an interface for medical personnel to view reading data via trend graphs and individual reading tracings; provide an interface for medical personnel to establish global thresholds for all patients; provide an interface for medical personnel to establish patient specific thresholds; provide an interface for medical personnel to annotate the trend graph with medication changes, notes, and hospitalization events.

In one aspect and despite the patient-centric application of the front end computer system 18, in some embodiments front end computer system 18 serves a purpose of applying a layer of clinically relevant information to the raw physiological data coming from the patient monitors 12. In particular, the front end computer system 18 can be configured to request and record a wealth of patient, diagnostic and other value-adding information that can be subsequently overlaid or otherwise associated with the unique physiological information being streamed from the patient monitors 12.

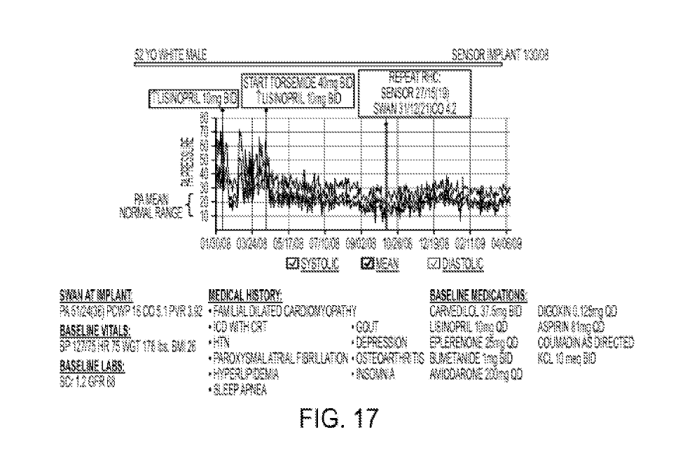

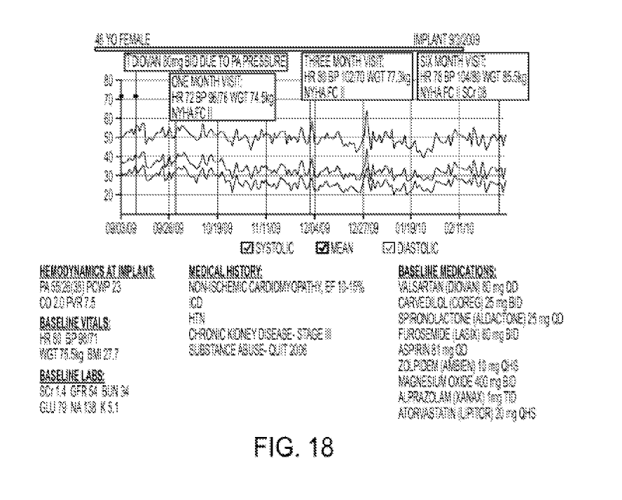

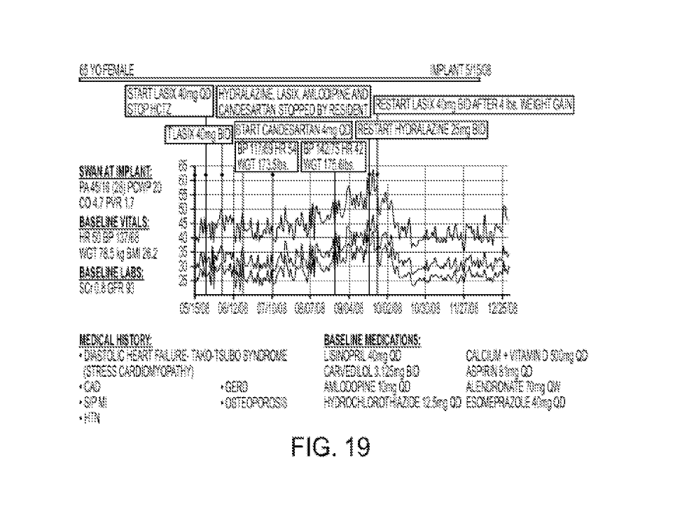

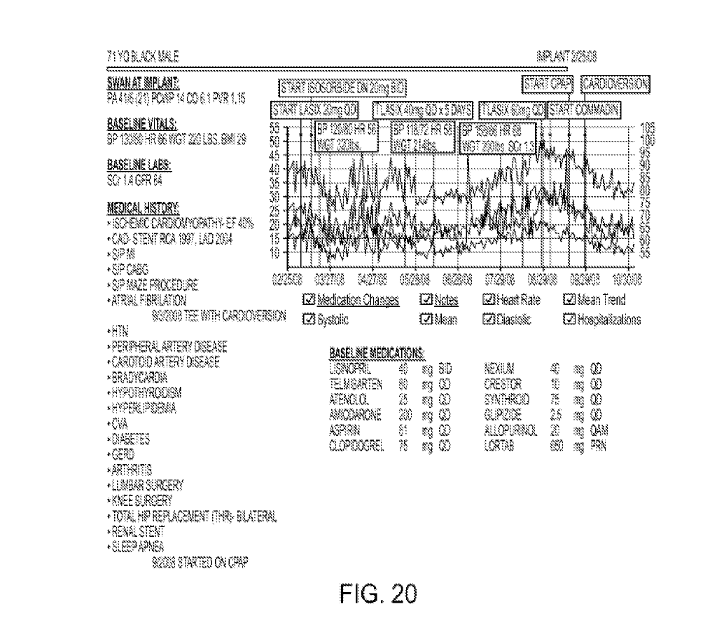

Examples of the high-value information that is entered by the healthcare personnel, associated with the physiological information and then sent for storage on the physiological information database 20 are illustrated by the individual patient cases as shown in FIGS. 16-20. Information collected and added at this stage can comprise at least one of patient profile and demographic information (age, race, gender, weight, and the like), medical history, medications, classifications, diagnoses, and the like. Other information can comprise at least one of patient episodes, such as surgeries, catheterizations, changes in weight or medication that are associated with a timestamp at entry, and the like. In one aspect, the front end computer system 18 can be configured to associate timestamps for the physiological information with the timestamps of the patient events. In one aspect, the front end computer system 18 can be configured to record information and events that are part of the healthcare personnel's therapeutic efforts and correlate that information with the physiological information received from the patient monitors 12.

In one aspect, the front end computer system 18 can be configured to set and/or request a set of alerts that are warning thresholds for each patient or a group of patients. For example, mean pressure below 10 mmHg or above 20 mmHg. Diastolic pressure below 8 mmHg or above 20 mmHg. Systolic pressure below 15 mm Hg or above 35 mmHg.

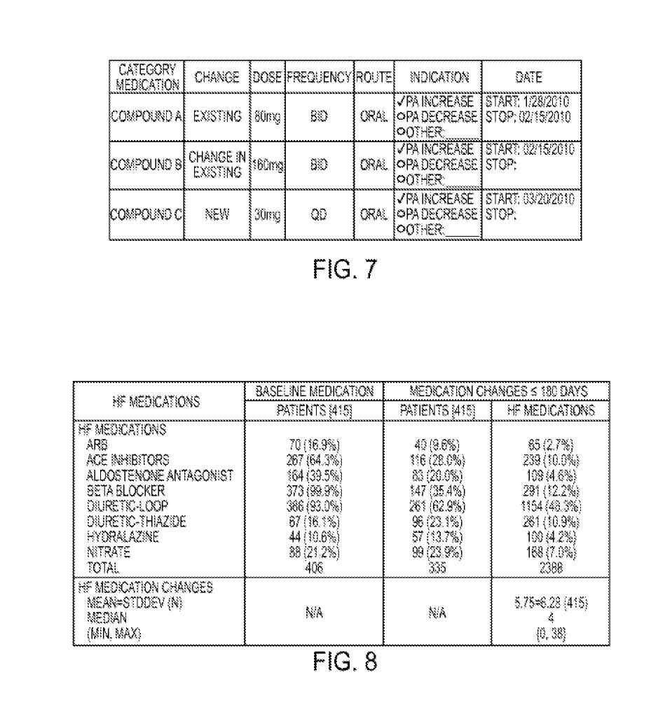

As one will appreciate, other options can comprise the creation and association of files or data that comprise trend graphs selected by the healthcare personnel 13. For example, trend lines can exemplarily be for systolic, diastolic, mean and pulse pressures, and their respective baselines. These selected trend lines can also be associated with start and stop timestamps, and the data can be superimposed on the raw physiological data to be stored on the database 20. FIG. 7, for example, shows entries of dosage information with associated timestamps that can be correlated with incoming streams of physiological information.

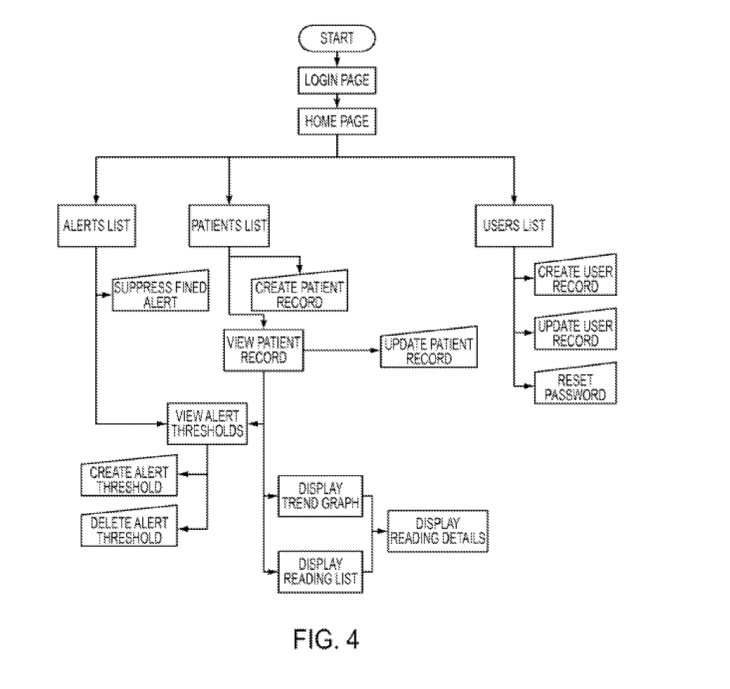

In one aspect, the front end computer system 18 of another embodiment of the present invention is shown in FIG. 4, which shows a flowchart of interactions between the front end computer system 18 and the patient monitors 12 and the healthcare personnel 13.

In another aspect, the backend computer system 19 can be configured, in the embodiment of FIG. 1, to act as an administrative portal for the service provider 22 to the information stored on the physiological information database 20. In one aspect, the backend computer system 19 can be configured to act as an access portal to the therapeutic investigator system 24, thereby enabling the therapeutic investigator system to conduct therapeutic development activities.

Exemplarily, both the service provider 22 and the therapeutic investigator system are shown being connected through a single cloud network 14, however it should be appreciated that the network 14 can comprise a plurality of separate networks. In one aspect, the service provider 22 can be physically resident nearby to the database 20 and the network 14 only a local-area network, while the Internet can serve as a longer-distance, more widely accessible network 14 for the therapeutic investigator. Regardless of the structure of the network 14, it is contemplated that all parties, including the submission, management and retrieval of the information on the physiological information database 20 extend through the security of the security system 16, which, in this embodiment and without limitation, can be a reverse proxy load balancer 16.

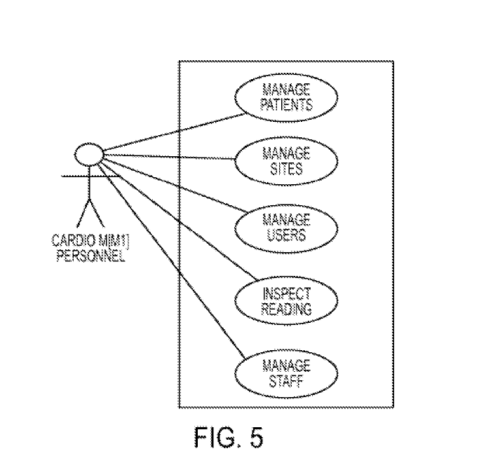

Turning to the embodiment shown in FIG. 5, the backend computer system 19 can be configured to perform the functions of managing patients, sites, users, inspecting readings and managing staff. In this embodiment, the backend computer system provides a mechanism for service providers 22 to view readings which have not been automatically bypassed based on the scoring algorithm. The reviewing personnel will be able to see detailed data for each reading as well as view the pressure waveform and signal strength plots. Approving a reading will cause a job to be queued to finish the processing asynchronously. Rejecting a reading will immediately transition the reading to its final state.

In various optional aspects, the back end computer system 19 can be configured to perform one or more of the following functions associated with the access provided to the service provider 22: allow service providers 22 with appropriate permissions to manage which users (patient monitors 12, therapeutic investigator systems 24, healthcare personnel 13, etc.) can access the system; allow service providers 22 to manage clinical investigation sites associated with one or more healthcare personnel 13 and their associated patient monitors 12; allow service providers 22 to manage users within a site; allow service providers 22 to view sensor records; allow service providers 22 to manually inspect readings that were not automatically accepted based on the automated scoring algorithm.

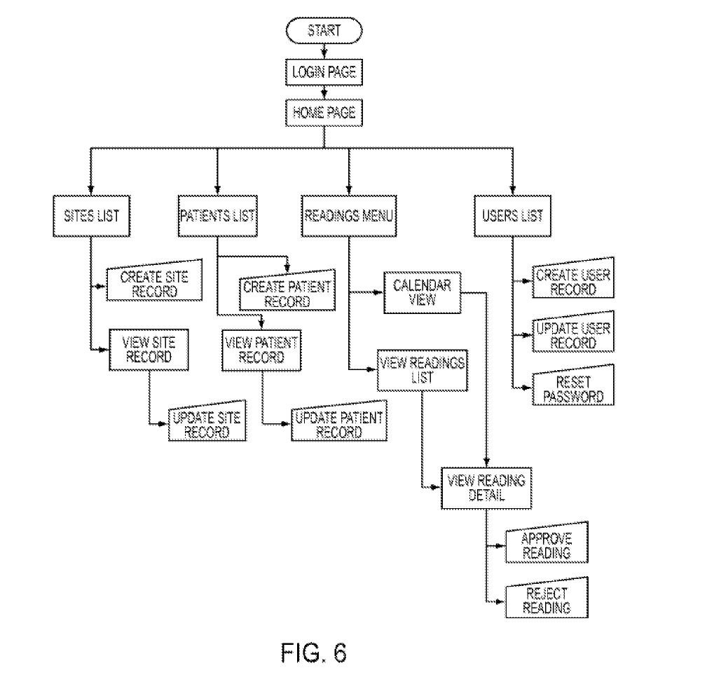

Turning now to FIG. 6, showing a flowchart of interactions between the back end computer system of another embodiment and the service providers 22 in their more administrative capacity for individual data receipt and database management. In other embodiments, the backend computer system 19 can be configured to interact with the therapeutic investigator system 24 to perform a range of functions and processes that provide physiological information from the database 20 for the development of therapeutics. For example and without limitation, the backend computer system 19 can be configured to support or implement a process for developing a therapeutic wherein the physiological information comprises cardiovascular physiology information. Such cardiovascular physiology information can comprise, for example and without limitation: hemodynamic monitoring information, pulmonary arterial pressure, cardiac output, peripheral vascular resistance, total peripheral resistance, heart rate, respiratory rate, dicrotic notch information, and the like. It is contemplated that such physiology information can be derived or otherwise obtained from conventional ocular, neurological, urological and gastroenterological systems.

In one aspect, the physiology information can be ambulatory information that is remotely obtained from patients outside of the hospital setting. For example, the desired physiology information could be obtained via a wireless sensor that's implanted in the patient's body, such as the exemplary CARDIOMEMS pressure sensor implanted in the patient's pulmonary artery. As one skilled in the art will appreciate, ambulatory data collection is supported if such a sensor lacks percutaneous connections that would stop or impede normal daily activities. In one aspect, the physiological information can be derived from a sensor that is passive and energized from an external source, such as, for example and without limitation, RF energy of an electromagnetic field.

In another aspect, the backend computer system 19 can be configured to prospectively guide development of the therapeutic using the database of physiological information 20. For example, the backend computer system 19 can facilitate design of the therapeutic by revealing compounds that have particular effects on the physiological information by studying correlations made by the front end system 18 between the dosage administration and the remotely collected, high-fidelity physiological information supplied by the monitoring systems 12. In one aspect, guiding development of the therapeutic can comprise designing a testing protocol for the therapeutic using trends and other information revealed from the database of physiological information 20, which could comprise identification of patients or patient characteristics making them particularly sensitive to therapeutics and therefore useful in clinical trials.

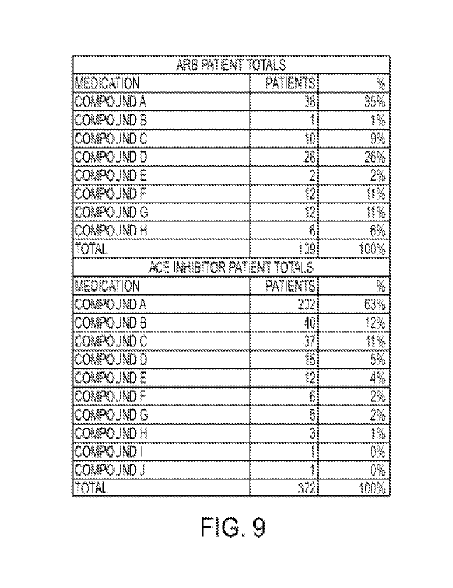

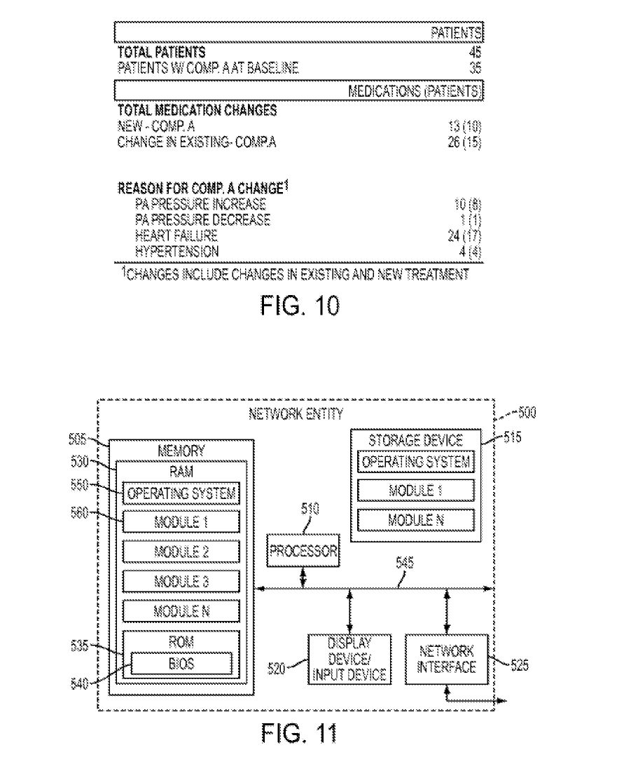

In one aspect, the backend computer system 19 can be configured to model predicted characteristics of a therapeutic, such as efficacy, drug-drug interaction, safety, adverse events or dosing. FIGS. 8-10 show data mined from the database of physiological information 20 using various aspects of the backend computer system 19.

In one aspect, the backend computer system 19 can be configured to facilitate meeting the requirements of a regulatory authority by providing access through the therapeutic investigator system 24 (or, even directly through the network 14) to the database of physiological information 20.

In one aspect, the backend computer system 19 can be configured for developing a therapeutic using a database of physiological information. For example, the physiological information can comprise cardiovascular physiology information such as, for example and without limitation, at least one of: hemodynamic monitoring information, pulmonary arterial pressure, cardiac output, peripheral vascular resistance, total peripheral resistance, heart rate, respiratory rate, dicrotic notch information, and the like. Optionally, the cardiovascular physiology information can also comprises ambulatory cardiovascular information. It is contemplated that such cardiovascular physiology information can be derived or otherwise obtained from conventional ocular, neurological, urological and gastroenterological systems.

The cardiovascular physiology information can in some aspects be remotely obtained. For example, the cardiovascular physiology information can be obtained wirelessly. In some examples, the cardiovascular information is obtained from an implanted sensor. The implanted sensor can be a pressure sensor. The pressure sensor can be implanted in a pulmonary artery.

In one aspect, the backend computer system 19 can be configured for development of a therapeutic, which can comprise prospectively guiding development of the therapeutic using a database of physiological information. The prospective guidance of development of the therapeutic can comprise designing the therapeutic and, optionally, can further comprise designing a testing protocol for the therapeutic. In various aspects, patients can be chosen for a clinical trial based on the physiological data. Furthermore, prospectively guiding can comprise modeling predicted characteristics of a therapeutic.

In one aspect, the backend computer system 19 can be configured for can optionally be used to predict characteristics of a therapeutic including at least one of efficacy, drug-drug interaction, safety, adverse events or dosing. Optionally, developing the therapeutic using the database comprises using the database to meet regulatory requirements.

In one aspect, the backend computer system 19 can be configured for predicting an effect of a candidate therapeutic agent on a hemodynamic parameter of a patient. The systems and methods can comprise providing at least one database including hemodynamic data, which can comprise a plurality of hemodynamic values measured in one or more subjects.

In one aspect, a candidate therapeutic agent for administration to a patient can be identified and all or a subset of the hemodynamic data can be correlated with the candidate therapeutic agent to indicate a predicted change in one or more hemodynamic values in the patient that would result from administration of the candidate agent. The predicted change can be used to indicate the predicted effect of the candidate agent on the hemodynamic parameter of the subject. In another aspect, the backend computer system 19 can be configured for designing therapeutic agents that comprise determining a change to a hemodynamic parameter of a subject or an expected change resulting from administration of the therapeutic agent. The change or expected change in the hemodynamic parameter can be used to design a therapeutic agent. For example, if the change or expected change is desirable, the therapeutic agent can be optionally modified to increase the magnitude, onset or duration of the change. Similarly, if the change or expected change is undesirable, the therapeutic agent can be optionally modified to decrease the magnitude, onset or duration of the change.

In one aspect, the backend computer system 19 can be configured for identifying a subject based on a specified hemodynamic response to a therapeutic agent. For example, characteristics of the subject that indicate an increased likelihood that the subject will have the specified hemodynamic responses can be determined. Optionally, the identified subject or a plurality of subjects having the same or similar determining characteristics can be selected to participate in a clinical study for the therapeutic agent.

In one aspect, the backend computer system 19 can be configured for developing a therapeutic agent or regimen for administering the therapeutic agent or for assessing the safety or efficacy of a therapeutic agent. For example, a change to a hemodynamic parameter of a subject or an expected change resulting from administration of the therapeutic agent can be determined. The change or expected change in the hemodynamic parameter can be used to develop the therapeutic agent or regimen or used to assess the efficacy of the therapeutic agent.

In one aspect, the backend computer system 19 can be configured for assessing an effect of a therapeutic agent on a hemodynamic parameter of a subject are also provided and comprise providing at least one database including hemodynamic data comprising a plurality of hemodynamic values measured in one or more subjects having been administered a therapeutic agent. In one aspect, a change in one or more of the measured hemodynamic values resulting from the administration of the therapeutic agent can be identified, the change indicating an effect of the therapeutic agent on the hemodynamic parameter of the subject. The hemodynamic data can comprises at least one hemodynamic value measured in a subject prior to administration of the therapeutic agent; at least one hemodynamic value measured in a subject concurrent with administration of the therapeutic agent; and/or at least one hemodynamic value measured in a subject subsequent to administration of the therapeutic agent. Optionally, the hemodynamic data comprises at least one hemodynamic value measured in a subject prior to administration of the therapeutic agent and at least one hemodynamic value measured in a subject subsequent to administration of the therapeutic agent. In some aspects, one or more additional therapeutic agents can be administered to the subject prior to, concurrently with, or subsequent to the therapeutic agent.

It is contemplated that the therapeutic agent can be modified to increase the indicated effect. For example, if the indicated effect is desired, the structure of the therapeutic agent can be modified to increase the indicated effect. The therapeutic agent can also be modified to decrease the indicated effect. Similarly, if the indicated effect is not desirable, then the structure of the therapeutic agent can be modified to decrease the indicated effect. Moreover, an administration characteristic of the therapeutic agent can be modified to increase or decrease the indicated effect. In one aspect, the administration characteristic can be selected from the group comprising at least one of: dosage amount, number of doses, timing of doses, route of administration, total dosage, and the like. When the indicated effect is to be increased or decreased, one or more portions of the therapeutic agent responsible for the indicated effect can be determined. Optionally, a second therapeutic agent including the one or more portions of the therapeutic agent responsible for the indicated effect can be designed.

In one aspect, the indicated effect can used to assess safety of the therapeutic agent for administration to a mammal or population thereof. In some examples, the indicated effect can be used to assess the toxicity, such as, for example and without limitation, cardiac toxicity, of the therapeutic agent for administration to a mammal or population thereof. The indicated effect can also be used to assess the efficacy of the therapeutic agent for administration to a mammal or population thereof. In one aspect, it is contemplated that the indicated effect can also be used to predict the effect or effects of the therapeutic agent or agents having the same or similar pharmacological characteristics on the hemodynamic parameter. Optionally, the indicated effect can be used to predict the effect or effects of the therapeutic agent or agents having the same or similar pharmacological characteristics on a hemodynamic parameter of a mammal. In some aspects, the indicated effect can be used to determine an end point for a clinical trial.

In one aspect, the method and system can further comprise determining one or more characteristic of the subject such as, for example and without limitation, a physical characteristic, a physiologic characteristic, a metabolic characteristic, a chronological characteristic, a disease state, a drug administration history, a medical history, and/or a genetic characteristic. In one aspect, the characteristic can be correlated with the indicated effect in the subject. In this aspect, the correlation of the characteristic and the indicated effect in the subject can be used to select one or more additional subjects for administration of the therapeutic agent or for a therapeutic agent having the same or similar indicated effect. Optionally, the correlation of the characteristic and the indicated effect can be used to select one or more additional subjects to participate in a clinical trial for the therapeutic agent or for a therapeutic agent having the same or similar indicated effect.

In one aspect, the backend computer system 19 could track therapy deployment after FDA allowance (i.e., while on the market) for whole populations, groups of patients or even individual patients. For example, dose titration could be personalized by modifying timing, dosage and mixtures of therapeutics such as drugs or treatment protocols. Also, the backend computer system 19 could be used for including, excluding or ceasing administration of a drug.

In one aspect, the correlation of the characteristic and the indicated effect in the subject is used to select or modify a therapeutic regimen in the subject or in another subject having the same or similar characteristics. Such selection or modification can comprise selecting or modifying drug administration protocol including dosage of one or more therapeutic agent, selection of one or more therapeutic agent, combination of therapeutic agents, and/or timing of administration of one or more therapeutic agent.

In a further aspect, the indicated effect can also be used to alter a treatment protocol of a subject. For example, the indicated effect can be used for determining whether to administer less of the therapeutic agent, administering more of the therapeutic agent, discontinuing use of the therapeutic agent, administering one or more additional agents, and the timing of administration of the agent.

It is contemplated in the methods and systems described herein, that the hemodynamic parameters can optionally be selected from the group comprising: heart rate, systolic blood pressure, diastolic blood pressure, mean blood pressure, stroke volume, cardiac output, peripheral vascular resistance, total peripheral resistance, pulmonary arterial pressure, and the like. It is further contemplated that the hemodynamic values measured in the subject can optionally be measured using an implantable sensor device, which can optionally measure hemodynamic parameters selected from the group comprising: heart rate, systolic blood pressure, diastolic blood pressure, mean blood pressure, stroke volume, cardiac output, peripheral vascular resistance, total peripheral resistance, pulmonary arterial pressure, and the like.

In one aspect, although the backend computer system 19 of FIG. 1 is shown as being a discrete computer system separate from the therapeutic investigator system 24 and the database 20, embodiments of the present invention can comprise the above-described functionality spread through these systems, with each performing some or all of the functions described and/or additional systems allocated at different locations and variably interconnected through networks 14. In one example, the backend computer system 19 and the database of physiological information 20 and other computer systems can be configured to cooperate to execute instructions to facilitate development of therapeutics. In this example, the computer systems can comprise a memory on which is stored the database 20 having high-fidelity physiological information obtained from a plurality of patient sensor systems 12 wherein the data is associated or correlated with a plurality of associated conditions entered at the front end computer system 18 by the healthcare personnel 13. As one will appreciate, instructions are provided on the memory of the computer systems that direct receiving from a user (such as therapeutic investigator system 24) an inquiry about a particular therapeutic. The inquiry can comprise a design inquiry for prospectively predicting success of the therapeutic based the predicted physiological impact of the therapeutic and the high-fidelity physiological information. Instructions can also be comprised that determine a relationship between the therapeutic and one of the associated conditions and/or the high-fidelity physiological information sorted on the database 20.

In another aspect, not only is the physiological information high-fidelity, but it is obtained from an implanted sensor collecting information while the patient is ambulatory. Also, instructions can be comprised on the memory for receiving a date stamp associated with the high-fidelity physiological information and with the ambulatory conditions and instructions for correlating the date stamps to develop associative information characterizing temporal relationships between the high-fidelity physiological information and the ambulatory conditions and store the associative information on the database.

In one aspect, the physiological information can be used in systems and methods for developing a therapeutic using a database of physiological information. For example, the physiological information can comprise cardiovascular physiology information, such as, for example and without limitation, at least one of: hemodynamic monitoring information, pulmonary arterial pressure, cardiac output, peripheral vascular resistance, total peripheral resistance, heart rate, respiratory rate, dicrotic notch information, and the like. Optionally, the cardiovascular physiology information can comprise ambulatory cardiovascular information. It is contemplated that such cardiovascular physiology information can be derived or otherwise obtained from conventional ocular, neurological, urological and gastroenterological systems.

The development of a therapeutic can comprise prospectively guiding development of the therapeutic using a database of physiological information. The prospective guidance of development of the therapeutic can comprise designing the therapeutic and, optionally, can further comprise designing a testing protocol for the therapeutic. In some aspects, patients can be chosen for a clinical trial based on the physiological data. Furthermore, prospectively guiding can comprise modeling predicted characteristics of a therapeutic.

In various aspect, the systems and methods can optionally be used to predict characteristics of a therapeutic including at least one of efficacy, drug-drug interaction, safety, adverse events, dosing, and the like. Optionally, developing the therapeutic using the database can comprise using the database to meet regulatory requirements.

In one aspect, the systems and methods can be configured to predict an effect of a candidate therapeutic agent on a hemodynamic parameter of a patient. The systems and methods can comprise providing at least one database including hemodynamic data, which hemodynamic data can comprise a plurality of hemodynamic values measured in one or more subjects.

In one aspect, a candidate therapeutic agent for administration to a patient can be identified and all or a subset of the hemodynamic data can be correlated with the candidate therapeutic agent to indicate a predicted change in one or more hemodynamic values in the patient that would result from administration of the candidate agent. The predicted change can be used to indicate the predicted effect of the candidate agent on the hemodynamic parameter of the subject. In another aspect, the systems and methods for designing therapeutic agents can comprise determining a change to a hemodynamic parameter of a subject or an expected change resulting from administration of the therapeutic agent. The change or expected change in the hemodynamic parameter can then be used to design a therapeutic agent. For example, the change or expected change is desirable and the therapeutic agent can be optionally modified to increase the magnitude, onset or duration of the change. In another example, the change or expected change is undesirable and the therapeutic agent can be optionally modified to decrease the magnitude, onset or duration of the change.

In another aspect, the systems and methods can also comprise identifying a subject based on a specified hemodynamic response to a therapeutic agent. For example, characteristics of the subject that indicate an increased likelihood that the subject will have the specified hemodynamic responses can be determined. In this aspect, the identified subject or a plurality of subjects having the same or similar determining characteristics can be selected to participate in or be excluded from a clinical trial or study for the therapeutic agent. Also, the systems and methods can screen populations to identify subpopulations for study that have a common profile characteristic such as age, weight, gender or genetic markers.

In a further aspect, the systems and methods can comprise developing a therapeutic agent or regimen for administering the therapeutic agent. In this aspect, a change to a hemodynamic parameter of a subject or an expected change resulting from administration of the therapeutic agent can be determined and can be used to develop the therapeutic agent or regimen.

In another aspect, the systems and methods can comprise assessing the safety or efficacy of a therapeutic agent. In this aspect, a change to a hemodynamic parameter of a subject or an expected change resulting from administration of the therapeutic agent can be determined. The determined change or expected change in the hemodynamic parameter can subsequently be used to assess the efficacy of the therapeutic agent.

In one aspect, the physiological information database 24 of the embodiment shown in FIG. 1 can comprises a robust collection of high-fidelity cardiovascular information that is associated with a range of medication data points including, for example and without limitation: medication name, category, dose, frequency, route, change (existing, new, change in existing), indication (PA increase, PA decrease, other), start/stop dates, and the like.

In one aspect, as can also be seen from FIG. 1, the front end system and back end system 18, 19 can share the database 24. In this embodiment, many of the same pieces of data can be manipulated in both systems; therefore, they can also share most of the model space.

Optionally and as one skilled in the art will appreciate, application software for managing embodiments of the database(s) described herein can employ a language such as structured query language (SQL). SQL is a database computer language designed for managing data in relational database management systems (RDBMS), and originally based upon relational algebra. Its scope comprises data insert, query, update and delete, schema creation and modification, and data access control. In another aspect, embodiments can employ PostgreSQL, for example, which is an open source object-relational database system particularly well-suited for use on a range of platforms including the aforementioned Linux-based operating system. It is relatively low-cost, makes for easy development and migrates easily between different operating system platforms. Such software could also be resident on one or more of the other systems 18, 19, 24, 26 to enable or enhance their ability to interact with the raw data on the database of physiological information 20.

As will be appreciated by one skilled in the art, aspects of the present invention can be embodied as a system, method or computer program product. Accordingly, aspects of the present invention can take the form of an entirely hardware embodiment, an entirely software embodiment (including firmware, resident software, micro-code, etc.) or an embodiment combining software and hardware aspects that can all generally be referred to herein as a “circuit,” “module” or “system.” Furthermore, aspects of the present invention can take the form of a computer program product embodied in one or more computer readable medium(s) having computer readable program code embodied thereon.

It is contemplated that any combination of one or more computer readable medium(s) can be utilized. In various aspects, the computer readable medium can be a computer readable signal medium or a computer readable storage medium. A computer readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would comprise the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium can be any tangible medium that can contain, or store a program for use by or in connection with an instruction execution system, apparatus, or device.

In one aspect, a computer readable signal medium can comprise a propagated data signal with computer readable program code embodied therein, for example, in baseband or as part of a carrier wave. Such a propagated signal can take any of a variety of forms, including, but not limited to, electro-magnetic, optical, or any suitable combination thereof. A computer readable signal medium can be any computer readable medium that is not a computer readable storage medium and that can communicate, propagate, or transport a program for use by or in connection with an instruction execution system, apparatus, or device. It is contemplated that the program code embodied on a computer readable medium can be transmitted using any appropriate medium, including but not limited to wireless, wireline, optical fiber cable, RF, etc., or any suitable combination of the foregoing.

In a further aspect, computer program code for carrying out operations for aspects of the present invention can be written in any combination of one or more programming languages, including an object oriented programming language such as Java, Smalltalk, C++ or the like and conventional procedural programming languages, such as the “C” programming language or similar programming languages. The program code can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions can also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions can also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Applications described herein can be implemented using various software languages, such as Linux. Linux is an open-source software preferred for servers and has the attributes, when applied to embodiments of the present invention of agility without sacrificing simplicity, stability or compatibility.

Web or internet applications described herein can be implemented using various web programming and application packages, such as Ruby on Rails (RoR). RoR is an open-source web application framework that uses the Ruby programming language. Ruby on Rails comprises tools that make common development tasks easier “out of the box”, such as scaffolding that can automatically construct some of the models and views needed for a basic website. RoR, for embodiments of the present invention, supplies code efficiency, a relatively short development cycle and it can be run on a JAVA server with Jruby.

Referring now to FIG. 11, a schematic diagram of a central server 500, or similar network entity, configured to develop a therapeutic using a database of physiological information, according to one embodiment of the invention, is provided. As used herein, the designation “central” merely serves to describe the common functionality the server provides for multiple clients or other computing devices and does not require or infer any centralized positioning of the server relative to other computing devices. As can be understood from FIG. 11, in this embodiment, the central server 500 can comprise a processor 510 that communicates with other elements within the central server 500 via a system interface or bus 545. Also comprised in the central server 500 can be a display device/input device 520 for receiving and displaying data. This display device/input device 520 can be, for example, a keyboard or pointing device that is used in combination with a monitor. The central server 500 can further comprise memory 505, which can comprise both read only memory (ROM) 535 and random access memory (RAM) 530. The server's ROM 535 can be used to store a basic input/output system 540 (BIOS), containing the basic routines that help to transfer information across the one or more networks.

In addition, the central server 500 can comprise at least one storage device 515, such as a hard disk drive, a floppy disk drive, a CD Rom drive, or optical disk drive, for storing information on various computer-readable media, such as a hard disk, a removable magnetic disk, or a CD-ROM disk. As will be appreciated by one of ordinary skill in the art, each of these storage devices 515 can be connected to the system bus 545 by an appropriate interface. The storage devices 515 and their associated computer-readable media can provide nonvolatile storage for a personal computer. It is important to note that the computer-readable media described above could be replaced by any other type of computer-readable media known in the art. Such media comprise, for example, magnetic cassettes, flash memory cards, digital video disks, and Bernoulli cartridges.

A number of program modules can be stored by the various storage devices and within RAM 530. Such program modules can comprise an operating system 550 and a plurality of one or more (N) modules 560. The modules 560 can control certain aspects of the operation of the central server 500, with the assistance of the processor 510 and the operating system 550. For example, the modules can perform the functions described above and illustrated by the figures and other materials disclosed herein.

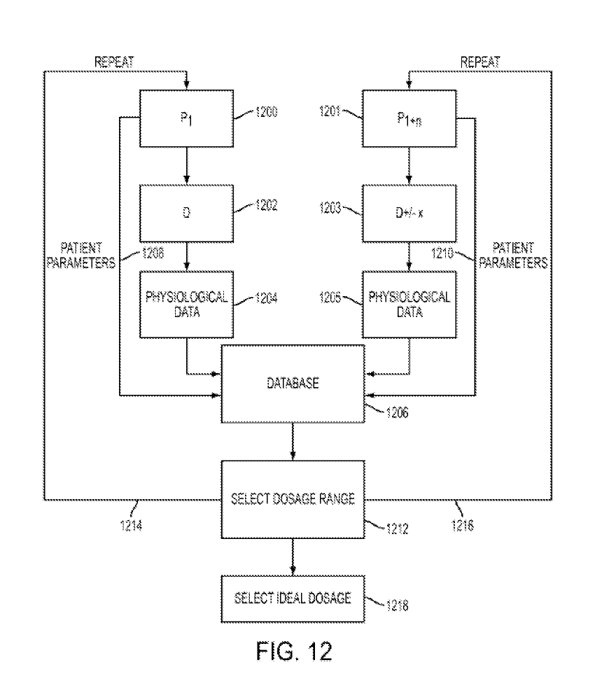

FIG. 12 illustrates an example method for developing a therapeutic. In steps 1200 and 1201 of the example method, at least two patients P1 and P1+n (wherein n=1, 2, 3 . . . ) are selected and these patients are administered a therapeutic agent in steps 1202 and 1203. For example, P1 is administered an agent at a dosage D and Px+1n is administered a dosage D+/−X, wherein X is zero or any number greater than zero. Thus, D and D+/−X are optionally different dosages of the same agent. In steps 1204 and 1205 physiological data resulting from the administered agents and the dosages and other patient information are collected from each patient and stored in a database in step 1206 as described above.

The physiological data collection and types of physiological data are described above and can optionally be, or comprise, hemodynamic data. As also described above, other features or patient parameters can be stored on the database as shown in the example method in steps 1208 and 1210, or on a database in communication with the database. The patient parameters can, for example, comprise but are not limited to age, weight, body mass index, disease state, medical history, family history, medication history, concurrent medications, sex, and the like. In step 1212, the physiological data, which is optionally combined with one or more patient parameters, can be used to select a desired dosage or dosage range for the agent.

The desired dosage or a dosage within the desired dosage range can be given to any of the patients P1 to P1+n, or, optionally, can be used to guide dosage decisions in other individuals of a patient population, that has not been monitored. After a desired dosage or dosage range is determined, the process steps (1200-1212) can be repeated as shown by steps 1214 and 1216 to further determine increasingly ideal dosages or dosage ranges for the agent in step 1218.

These determinations can be used to facilitate development of a therapeutic by efficiently identifying preferred dosages that are correlated to improved physiological data for clinical trials. The determinations can also be used to for determining proper dosages of commercial products for general and specific populations of subjects. For example, the method can be used to arrive at dosing levels based on patient/subject profiles including, but not limited to, pulmonary artery pressure response to a study or commercial drug or with other hemodynamic metrics alone or in combination with characteristics such as age, weight and concurrent drug administration or drug-drug interaction.

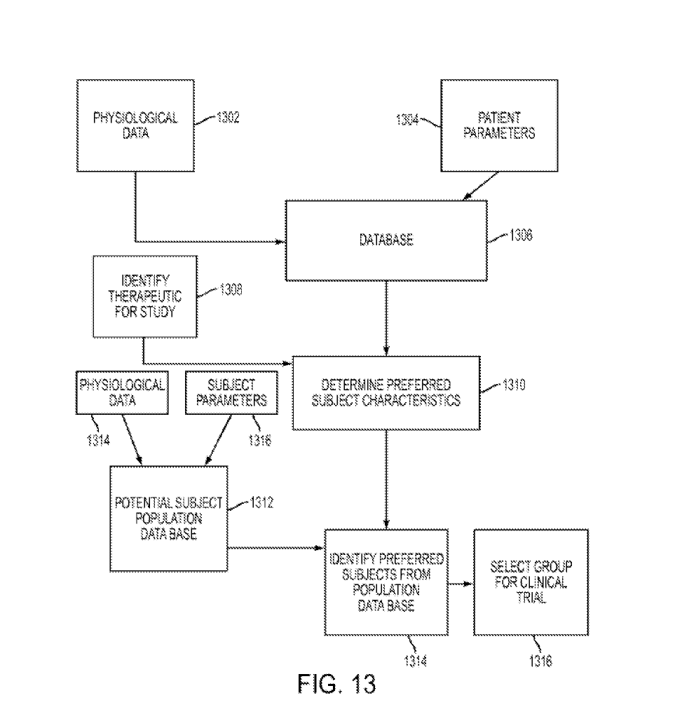

FIG. 13 illustrates another example method in accordance with the described invention. In this example method, patients, or a population of patients, are identified and optionally selected for a clinical trial for a given therapeutic. In this example, a database is provided in step 1306 that comprises patient physiological data gathered in step 1302, and that optionally comprises patient parameter data including patient treatment history gathered in step 1304. For example, these patient parameters or features comprise, but are not limited to, those parameters and features described throughout, such as age, weight, body mass index, disease state, medical history, family history, medication history, concurrent medications, sex, and the like.

In step 1308, the method further comprises selecting a therapeutic to study in a clinical trial or investigation. In step 1310, and based on the selected therapeutic, the database is interrogated for preferred subject characteristics based on a response or likely response to the selected therapeutic. For example, the safety or efficacy, or likely safety or efficacy, of the selected therapeutic in individuals or populations of individuals having certain identified characteristics can be determined.

In one aspect, the determined subject characteristics can then be compared with potential subject data to identify subjects that can have similar response to the therapeutic. For example, a second database of potential subjects can be provided in step 1312 that comprises physiological data gathered in step 1314, and optionally, patient parameters gathered in step 1316 that are the same or similar to the first database. The data for these subjects can be compared to the determined preferred subject characteristics in step 1314 to identify preferred subjects for the clinical trial from the potential subject population. One or more of the identified preferred subjects can be selected for the clinical trial in step 1316. Conversely, subjects not identified as having the preferred subject characteristics can be excluded from the clinical trial.

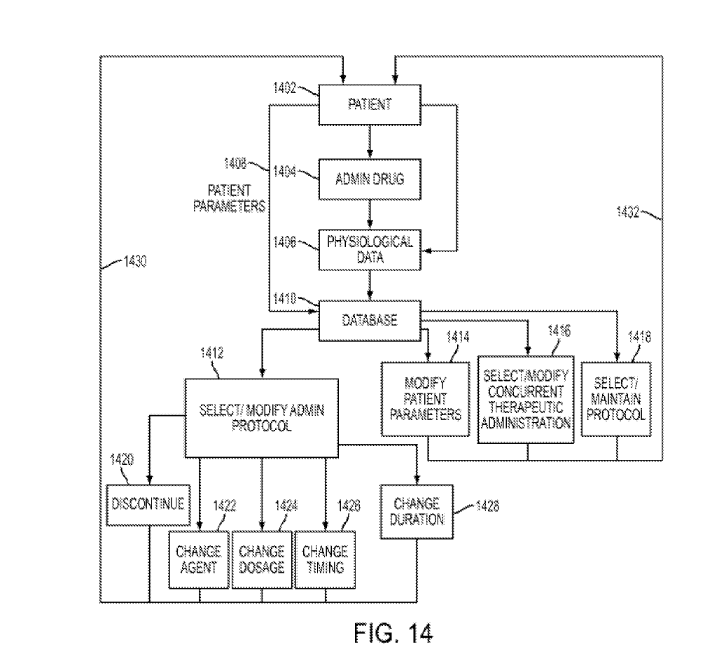

FIG. 14 illustrates yet another example method in accordance with the described invention. In this example, the use of a therapeutic for a patient is facilitated. In this example method, a patient 1402 is monitored in step 1406 to collect physiological data. Optionally, the physiological data is collected subsequent to administration of a therapeutic as shown in step 1404. The physiological data is stored in a database as described above as shown in step 1410. The physiological data can be collected as described above, and optionally are, or comprise, hemodynamic data. In addition to the physiological data, patient parameters including therapeutic administration history can be collected from the patient in step 1408 and stored in the database, or in one or more database in communication with the database storing the physiological information.

In various aspects, the patient parameters can comprise, but are not limited to, those parameters described throughout, such as age, weight, body mass index, disease state, medical history, family history, medication history, concurrent medications, sex, and the like. The stored data can be used to determine whether a therapeutic should be administered to the patient and/or whether a modification should be made to the patient's therapeutic regimen in steps. For example, through processing the data it can determined whether to administer a therapeutic or modify and administration protocol as shown in step 1412. For example, the step 1412 can comprise sub-steps 1420-1428 which are to discontinue a therapeutic 1420, change a therapeutic 1422, change the dosage of a therapeutic 1424, change the timing of administration of a therapeutic 1426, or to change the duration of administration of a therapeutic 1428.

Moreover, other factors that can alter the therapeutics' effect on the patient can be implemented or modified as shown in step 1414. For example, one or more patient parameter can be modified by the subject's incorporation of lifestyle changes (e.g. diet change, sleep pattern change). Furthermore, other therapeutics or therapeutic regimens can be implemented or modified as shown in step 1416. The therapeutics' use can also be facilitated by a determination to maintain any current protocol or patient parameters of the patient as shown in step 1418. The process steps can be repeated as shown by steps 1430 and 1432.

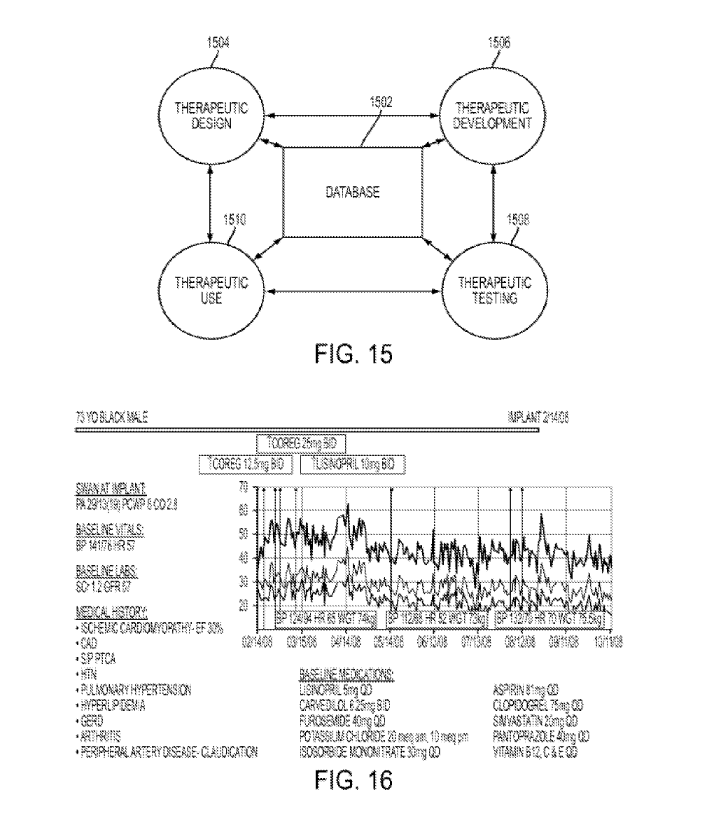

Referring now to FIG. 15, systems and methods described herein can be used to integrate pharmaceutical applications and to enhance overall efficiency of the pharmaceutical industry. For example, the above described database comprising physiological data, and optionally, patient parameters, can be used to integrate therapeutic design, therapeutic development, therapeutic testing and therapeutic use. In this regard, the stored data shown in the database 1502 can be communicated and used to make decisions that affect the design 1504, development 1506, testing 1508 and use 1510 of commercial and investigational drugs and their active ingredients. Moreover, as shown in FIG. 15, the information and decisions determined in each of these areas can be integrated with one or more other areas to provide overall enhancement of the pharmaceutical industry's efficiency in bringing safe and effective drugs to patients and patient populations.

Exemplary advantages of the embodiments of the methods and systems described herein comprise cost savings realized from earlier screening out of bad drug candidates. Non-invasive monitoring sensors, such as the CARDIOMEMS pressure sensor, provide for easier recruitment. And, the increased data per patient reduces the number of patients needed to demonstrate statistically significant outcomes. Additionally, combination therapeutics can be evaluated based on their physiological effects.

Thus, while there have shown and described and pointed out fundamental novel features of the invention as applied to preferred embodiments thereof, it will be understood that various omissions and substitutions and changes in the form and details of the methods described and devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention.

Moreover, it should be recognized that structures and/or elements and/or method steps shown and/or described in connection with any disclosed form or embodiment of the invention may be incorporated in any other disclosed or described or suggested form or embodiment as a general matter or design choice.

What is claimed is:

1. A process comprising:
developing a therapeutic using a database of physiological information, wherein the physiological information comprises cardiovascular physiology information, and wherein the cardiovascular physiology information comprises at least one of: hemodynamic monitoring information, pulmonary arterial pressure, cardiac output, peripheral vascular resistance, total peripheral resistance, heart rate, respiratory rate, and dicrotic notch information.

2. The process of claim 2, wherein the cardiovascular physiology information is remotely obtained.

3. The process of claim 2, wherein the cardiovascular physiology information comprises ambulatory cardiovascular information.

4. The process of claim 3, wherein the cardiovascular physiology information is obtained wirelessly.

5. The process of claim 4, wherein the cardiovascular information is obtained from an implanted sensor.

6. The process of claim 5, wherein the implanted sensor is a pressure sensor.

7. The process of claim 6, wherein the pressure sensor is implanted in a pulmonary artery.

8. The process of claim 5, wherein the sensor is a passive sensor configured to be energized by an electromagnetic field produced from an external source.

9. The process of claim 1, wherein developing further comprises prospectively guiding development of the therapeutic using the database of physiological information.

10. The process of claim 9, wherein prospectively guiding development of the therapeutic comprises designing the therapeutic.

11. The process of claim 10, wherein prospectively guiding development of the therapeutic further comprises designing a testing protocol for the therapeutic.

12. The process of claim 10, wherein prospectively guiding development of the therapeutic further comprises identifying patients for clinical trials.

13. The process of claim 10, wherein prospectively guiding comprises modeling predicted characteristics of the therapeutic.

14. The process of claim 13, wherein the predicted characteristics comprise at least one of: efficacy, drug-drug interaction, safety, adverse events, and dosing.

15. The process of claim 1, wherein developing the therapeutic using the database further comprises using the database to meet regulatory requirements and providing access to the physiological information on the database to a regulatory authority.

16. A computer system comprising a memory on which is stored:
a database including high-fidelity physiological information obtained from a plurality of patients and correlated with a plurality of associated conditions;
instructions for receiving from a user an inquiry about a therapeutic;
instructions for determining a relationship between the therapeutic and one of the associated conditions or the high-fidelity physiological information.

17. The computer system of claim 16, wherein the high-fidelity physiological information is obtained from an implanted sensor.

18. The computer system of claim 17, wherein the associated conditions are ambulatory conditions.

19. The computer system of claim 18, further comprising instructions for receiving a date stamp associated with the high-fidelity physiological information and with the ambulatory conditions and instructions for correlating the date stamps to develop associative information characterizing temporal relationships between the high-fidelity physiological information and the ambulatory conditions and store the associative information on the database.

20. The computer system of claim 18, wherein the inquiry about the therapeutic is a design inquiry configured to prospectively predict success of the therapeutic based on a predicted physiological impact of the therapeutic and the high-fidelity physiological information.

21. A method for assessing an effect of a therapeutic agent on a hemodynamic parameter of a subject, comprising:
providing at least one database including hemodynamic data comprising a plurality of hemodynamic values measured in one or more subjects having been administered a therapeutic agent; and
identifying a change in one or more of the measured hemodynamic values resulting from the administration of the therapeutic agent, the change indicating an effect of the therapeutic agent on the hemodynamic parameter of the subject.

22. The method of claim 21, wherein the hemodynamic data comprises at least one hemodynamic value measured in a subject prior to administration of the therapeutic agent.

23. The method of claim 21, wherein the hemodynamic data comprises at least one hemodynamic value measured in a subject concurrent with administration of the therapeutic agent.

24. The method of claim 21, wherein the hemodynamic data comprises at least one hemodynamic value measured in a subject subsequent to administration of the therapeutic agent.

25. The method of claim 21, wherein the hemodynamic data comprises at least one hemodynamic value measured in a subject prior to administration of the therapeutic agent and at least one hemodynamic value measured in a subject subsequent to administration of the therapeutic agent.

26. The method of claim 21, wherein one or more additional therapeutic agents are administered to the subject prior to, concurrently with, or subsequent to the therapeutic agent.

27. The method of claim 21, further comprising modifying the therapeutic agent to selectively increase or decrease the indicated effect.

28. The method of claim 21, further comprising modifying an administration characteristic of the therapeutic agent to selectively increase or decrease the indicated effect, wherein the administration characteristic is selected from the group consisting of: dosage amount, number of doses, timing of doses, route of administration, and total dosage.

29. The method of claim 21, further comprising determining one or more portions of the therapeutic agent responsible for the indicated effect.

30. The method of claim 29, further comprising designing a second therapeutic agent including the one or more portions of the therapeutic agent responsible for the indicated effect.

31. The method of claim 29, further comprising designing a second therapeutic agent wherein the one or more portions of the therapeutic agent responsible for the indicated effect are removed or reduced in effect.

32. The method of claim 21, wherein the indicated effect is used to assess safety of the therapeutic agent for administration to a mammal or population thereof.

33. The method of claim 21, wherein the indicated effect is used to assess the toxicity of the therapeutic agent for administration to a mammal or population thereof.

34. The method of claim 21, wherein the indicated effect is used to assess the efficacy of the therapeutic agent for administration to a mammal or population thereof.

35. The method of claim 21, wherein the indicated effect is used to predict the effect or effects of the therapeutic agent or agents having the same or similar pharmacological characteristics on the hemodynamic parameter.

36. The method of claim 21, wherein the indicated effect is used to predict the effect or effects of the therapeutic agent or agents having the same or similar pharmacological characteristics on a hemodynamic parameter of a mammal.

37. The method of claim 21, wherein the indicated effect is used to determine an end point for a clinical trial.

38. The method of claim 21, further comprising determining one or more characteristic of the subject, wherein the characteristic is selected from the group consisting of: a physical, physiologic, metabolic, chronological, disease state, drug administration history, medical history, and genetic characteristic.

39. The method of claim 38, wherein the characteristic is correlated with the indicated effect in the subject.

40. The method of claim 39, wherein the correlation of the characteristic and the indicated effect in the subject is used to select one or more additional subjects for administration of the therapeutic agent or for a therapeutic agent having the same or similar indicated effect.

41. The method of claim 39, wherein the correlation of the characteristic and the indicated effect is used to select one or more additional subjects to participate in a clinical trial for the therapeutic agent or for a therapeutic agent having the same or similar indicated effect.

42. The method of claim 39, wherein the correlation of the characteristic and the indicated effect in the subject is used to select or modify a therapeutic regimen in the subject or in another subject having the same or similar characteristics, wherein the selecting or modifying comprises selecting or modifying drug administration protocol including dosage of one or more therapeutic agent, selection of one or more therapeutic agent, combination of therapeutic agents, or timing of administration of one or more therapeutic agent.

43. The method of claim 21, further comprising using the indicated effect to alter the treatment protocol of the subject, wherein the alteration is selected from the group consisting of: administering less of the therapeutic agent, administering more of the therapeutic agent, discontinuing use of the therapeutic agent, administering one or more additional agents, and the timing of administration of the agent.

44. The method of claim 21, wherein the hemodynamic parameters are selected from the group consisting of heart rate, systolic blood pressure, diastolic blood pressure, mean blood pressure, stroke volume, cardiac output, peripheral vascular resistance, total peripheral resistance and pulmonary arterial pressure.

45. The method of claim 44, wherein the hemodynamic values measured in the subject are measured using an implantable sensor device.

46. The method of claim 45, wherein the implantable sensor device measures pulmonary arterial pressure and is implanted in the pulmonary artery, and wherein the implantable sensor communicates measurements of pulmonary arterial pressure remotely wirelessly.

47. A method for predicting an effect of a candidate therapeutic agent on a hemodynamic parameter of a patient, comprising:
providing at least one database including hemodynamic data comprising a plurality of hemodynamic values measured in one or more patients;
identifying a candidate therapeutic agent for administration to the one or more patients; and
correlating all or a subset of the hemodynamic data with the candidate therapeutic agent to indicate a predicted change in one or more hemodynamic values in the one or more patients that would result from administration of the candidate agent, the predicted change indicating the predicted effect of the candidate agent on the hemodynamic parameter of the one or more patients,
administering a therapeutic agent to the one or more patients, wherein the therapeutic agent administered to the one or more patients is of the same class as the candidate agent.

48. A method for designing a therapeutic agent, comprising:
determining a change to a hemodynamic parameter of a subject or an expected change resulting from administration of the therapeutic agent, and
using the change or expected change in the hemodynamic parameter to design a therapeutic agent,
wherein the change or expected change is desirable and the therapeutic agent is modified to increase the magnitude, onset or duration of the change or wherein the change or expected change is undesirable and the therapeutic agent is modified to decrease the magnitude, onset or duration of the change.

49. A method of identifying a subject based on a specified hemodynamic response to a therapeutic agent, comprising:
determining characteristics of the subject that indicate an increased likelihood that the subject will have the specified hemodynamic responses; and
selecting the identified subject or a plurality of subjects having the same or similar determining characteristics to participate in a clinical study for the therapeutic agent.

50. A method of developing a therapeutic agent or regimen for administering the therapeutic agent, comprising:
determining a change to a hemodynamic parameter of a subject or an expected change resulting from administration of the therapeutic agent; and
using the change or expected change in the hemodynamic parameter to develop the therapeutic agent or regimen.

51. A method for assessing the efficacy of a therapeutic agent, comprising:
determining a change to a hemodynamic parameter of a subject or an expected change resulting from administration of the therapeutic agent; and
using the change or expected change in the hemodynamic parameter to assess the efficacy of the therapeutic agent.
